(19) United States
(12) Patent Application Publication
(10) Pub. No.: US 2015/0313939 A1
(43) Pub. Date: Nov. 5, 2015

(54) ORAL IRON (III) BASED PHOSPHATE ADSORBENT FOR TREATING IRON-DEFICIENCY ANEMIA IN CATS WITH CHRONIC KIDNEY DISEASE

(71) Applicant: Jonathan King, Bennwil (CH)

(72) Inventor: Jonathan King, Bennwil (CH)

(21) Appl. No.: 14/649,264

(22) PCT Filed: Dec. 19, 2013
(86) PCT No.: PCT/EP2013/077393

§ 371 Date: Jun. 3, 2015

(30) Foreign Application Priority Data
Dec. 20, 2012 (EP) 12198690.5

(51) Int. Cl. A61K 33/26

(52) U.S. Cl. CPC A61K 33/26

(57) ABSTRACT

The invention relates to the new use of an oral iron(III)-based phosphate adsorbent, for example SBR759, in the treatment of iron deficiency anaemia in cats with chronic kidney disease.

There is significant evidence that maintaining phosphate balance is critical in healthy cats and especially in those that have disorders of phosphate metabolism, such as in chronic kidney disease. Phosphate concentrations in the plasma are regulated by complicated homeostatic mechanisms. Ultimately, regulation depends on the balance between dietary phosphate intake and excretion of surplus phosphate via the kidneys. Reduction in the excretory function of the kidney as chronic kidney disease progresses leads to an increase in plasma phosphate concentrations unless oral intake of phosphate is reduced.

Restriction of oral intake of phosphate in cats is achieved via two methods:

1) Diets containing low amounts of phosphate (‘renal diet’). The principle limitation of these prescription diets is that they are not palatable to many cats and maintenance of feed intake is essential in cats with chronic kidney disease.

2) Oral phosphate binders. These are administered with feed in order to bind phosphate both in the feed and in the lumen of the gastrointestinal tract. Phosphate binders are typically used in combination with a renal diet. However, phosphate binders can also be administered to cats that will not eat a renal diet and are fed instead a normal diet. A number of phosphate binders are registered for use in humans (reviewed by Tonelli et al., 2010). The older ones are based on calcium carbonate, calcium acetate and aluminium-based compounds. However, their use in cats is limited as a result of poor palatability and the potential for toxicity secondary to hypercalcaemia, for calcium-based products, and excessive aluminium for aluminium-based products. Two oral phosphate binders currently are marketed for use in cats: Ipakatine® (calcium carbonate) and Renalzin® (lanthanum carbonate octahydrate). Ipakatine® is sold as a nutraceutical in the EU without registration. Lantharenol® or Renalzin® is registered in the EU as a Food Additive under the category “other zootechnical additives”.

Until relatively recently, it was assumed that the benefit of phosphate binders was due only to restricting oral absorption of phosphate. However, a recent study concluded that benefits of phosphate binders are independent of baseline phosphate and treatment phosphate concentrations in human dialysis patients (Isakova et al., 2009). This evidence suggests there may be beneficial actions of phosphate binders other than binding phosphate, including binding of toxins in the gastrointestinal tract, reduction in levels of fibroblast growth factor (FGF)-23, and in the case of Fe-based phosphate binders, supplementation of dietary Fe (Isakova et al., 2009).

Anemia is present in 30-65% of cats with chronic kidney disease and is an independent risk factor for death or euthanasia (Chakrabarti et al., 2012). The results of this study indicated that proteinuria and hyperphosphatemia also were independent predictors of progression of feline chronic kidney disease. Furthermore, King et al. (2007) showed in studies with cats with chronic kidney disease that several baseline variables were significantly associated with a shorter renal survival time, including increased plasma concentration of creatinine, phosphate, and urea, increased urine protein-to-creatinine ratio, decreased blood hemoglobin concentration and hematocrit and increased blood leukocyte count. The pathogenesis of anemia secondary to progressive renal disease is multifactorial but the main mechanism involves reduced production of erythropoietin, a renal hormone that controls bone marrow production of red blood cells (Chalhoub et al. 2011). Although erythrocyte-stimulating agents can be used to counteract the effects of decreased erythropoietin production by the kidneys, their use is associated with complications such as iron deficiency, hypertension, arthralgia, fever, seizures, polycythemia and pure red cell aplasia (Chalhoub et al. 2011). Accordingly, there is a strong unmet need for treatments that improve red cell variables and quality of life in cats with chronic kidney disease.

SBR759 is a novel non-calcium, non-aluminium, iron (III)-based phosphate adsorbent. It is a complex produced from the addition of iron (III) chloride to potato starch, sucrose and sodium carbonate in water. Based on the single components of the complex, the following molecular formula can be written: [Fe5(OH)O7×4H2O]×sucrose×starch×hydrogen carbonate. It is insoluble and binds to inorganic phosphate. It is characterized by a phosphate binding capacity of at least about 120 mg adsorbed phosphate by 1 g of phosphate adsorbent, preferably of about 140 mg adsorbed phosphate by 1 g of phosphate adsorbent as described in International patent publication WO2008/071747. It is known and commercially available under the tradename Lenziaren™.

Recently, it was reported that the oral administration of SBR759 significantly reduced plasma phosphate concentrations, was well tolerated and had good palatability when administered to cats with chronic kidney disease and concomitant hyperphosphatemia. The optimal dose range was 0.25-1 g/day and it was well tolerated when administered in feed at doses of 0.5 to 2.0 g/day. Moreover, dose-related increases from baseline in plasma iron concentrations were observed in cats with chronic kidney disease and hyperphosphataemia, in particular at doses ≧0.5 g SBR759/day (Kuntz et al., 2012; Speranza et al., 2012).

Further examples of iron (III)-based phosphate binders are cross-linked dextran, i.e. iron(III)oxide-hydroxide-modified dextran (Hergesell & Ritz 1999); PA21, an iron-based phosphate adsorbent containing a mixture of polynuclear iron(III)-oxyhydroxide, starch and sucrose from Vifor and described in WO2006000547 (Geisser and Philipp, 2010; Wüthrich et al., 2012); fermagate, a non-calcium iron-magnesium hydroxycarbonate (McIntyre et al., 2009); and ferric citrate, also known as Zerenex™ from Keryx Biopharmaceuticals, Inc. and described in U.S. Pat. No. 6,903,235B (Sinsakul et al., 2012; Yokoyama et al., 2012).

It has now been surprisingly found that oral iron (III)-based phosphate adsorbents, for example SBR759, result not only in dose-related increases in plasma iron concentrations in cats with chronic kidney disease and hyperphosphataemia but also an improvement in the red cell variables, red cell count, haematocrit and haemoglobin concentration. In a further aspect, it has been found that oral iron (III)-based phosphate adsorbents, for example SBR759, significantly improve the quality of life scores of the cats and survival times are significantly longer compared to an historical control group.

This effect of oral iron (III)-based phosphate adsorbents, for example SBR759, on improving haematology variables may be due to improved renal function secondary to reduced plasma phosphate concentrations and/or due to increases in, and potentially normalization of, body iron concentrations.

Accordingly, in one aspect the present invention relates to an oral iron (III)-based phosphate adsorbent, for example SBR759, for use in a method of treating iron-deficiency anaemia in cats with chronic kidney disease. In a further aspect, the invention relates to an oral iron (III)-based phosphate adsorbent, for example SBR759, for use in a method of improving red cell variables in cats with chronic kidney disease.

In yet a further aspect, the invention relates to an oral iron (III)-based phosphate adsorbent, for example SBR759, for use in a method of increasing quality of life of cats with chronic kidney disease.

In yet a further aspect, the invention relates to an oral iron (III)-based phosphate adsorbent, for example SBR759, for use in a method of increasing survival in cats with chronic kidney disease.

In yet a further aspect, the invention relates to a method of treating iron-deficiency anaemia in cats with chronic kidney disease which method comprises administering an oral iron (III)-based phosphate adsorbent, for example SBR759, in a therapeutically effective amount, for example at a dose of 0.125 g to 10.0 g, e.g. 0.25 g to 1, 1.5, 2 or 5 g, per cat per day, for example wherein the oral iron (III)-based phosphate adsorbent, for example SBR759, dose is mixed homogenously into the daily food ration, for example, wherein the oral iron (III)-based phosphate adsorbent, for example SBR759, is administered with cat food continuously throughout the cat's life.

Oral iron (III)-based phosphate adsorbents, for example SBR759, are a useful addition to the available phosphate binders for use in cats. Advantages of oral iron (III)-based phosphate adsorbents, for example SBR759, compared to older phosphate binders for cats include:
High, irreversible phosphate binding potential
High palatability
Do not contain calcium, thereby reducing the risk of hypercalcemia and extraosseous calcification.
Do not contain aluminium, which can accumulate in bone and have toxic effects on bone (osteomalacia), brain (encephalopathy) and hematopoietic systems.
Do not contain lanthanum, which has suboptimal palatability and can cause gastrointestinal adverse events and may deposit in bone (Nunamaker & Sherman, 2011).
Contain iron, which according to the present invention may be beneficial in iron-deficiency anaemia.

Examples of iron (III)-based phosphate adsorbents useful according to the present invention include but are not limited to one or more of the following: SBR759, cross-linked dextran, PA21, fermagate, and iron salts such as ferric citrate, ferric chloride or ferric ammonium citrate. Preferably SBR759 may be used.

In a further aspect of the invention, one or more of the above iron (III)-based phosphate adsorbents may be added to available phosphate binders for use in mammals, e.g. companion animals, e.g. cats.

SBR759 is comprised of 22% (range 21-24%) iron in the form of ferric-oxide-hydroxide which is incorporated into a polymer of starch-sucrose to form a complex. The SBR759 molecule is designed to remain in the lumen of the gastrointestinal tract and to undergo no relevant absorption. This is due to its combination of low solubility and binding to phosphate in the lumen of the gastrointestinal tract preventing its absorption. However, in some situations small amounts of iron (Fe³⁺) can be released from the SBR759 and subsequently absorbed.

SBR 759 effectively binds phosphate and as a consequence results in significant decreases in serum phosphate at doses of 0.25, 0.5, 1.0 and 1.5 g/day. SBR759 is well tolerated and has good palatability in a population of cats with chronic kidney disease and hyperphosphatemia.

The palatability of SBR759 was shown to be good, and was significantly superior to the reference product Renalzin in healthy cats receiving a renal diet.

The main benefit of oral iron (III)-based phosphate adsorbents, for example SBR759, in cats derives from binding of phosphate in the diet and gastrointestinal tract, thereby assisting normalization of plasma phosphate concentrations. A secondary benefit is supplementation of dietary iron, which according to the present invention, is useful in cats with anemia secondary to chronic kidney disease.

These and other features, advantages and objectives of the present invention will be further understood and appreciated by those skilled in the art by references to the following specification and claims.

As used herein, the term iron (III)-based phosphate adsorbent may be used interchangeably with iron (III)-based phosphate binder.

As used herein, the term “iron (III)-based phosphate adsorbent” means any compound, composition, substance, medicament, drug, feed additive or active ingredient, for example, SBR759, cross-linked dextran, PA21, fermagate, ferric citrate, ferric chloride or ferric ammonium citrate, having a therapeutic or pharmacological effect, and which is suitable for administration to a mammal, e.g. a companion animal, e.g. a cat. Such iron (III)-based phosphate adsorbent should be administered in a “therapeutically effective amount”.

As used herein, the term “therapeutically effective amount” refers to an amount or concentration which is effective in reducing, eliminating, treating, preventing or controlling the symptoms of a disease or condition affecting the mammal. The term “controlling” is intended to refer to all processes wherein there may be a slowing, interrupting, arresting or stopping of the progression of the diseases and conditions affecting the mammal. However, “controlling” does not necessarily indicate a total elimination of all disease and condition symptoms, and is intended to include prophylactic treatment.

The appropriate therapeutically effective amount is known to one of ordinary skill in the art as the amount varies with the companion animal treated and the condition which is being addressed.

SBR759 is prepared by the addition of iron (III) chloride hexahydrate to starch, sodium carbonate and sucrose. At the end of the addition, the solids formed are isolated, washed and re-suspended in a mixture of ethanol and water. The suspension is finally dried to result in the active substance, which is filled into individual stickpacks to form the animal feed additive SBR759.

More specifically, SBR759 may be produced according to a process disclosed in International patent publication WO 2008/071747, which is hereby incorporated by reference.

For example, SBR59 may be produced according to any one of the following processes:

1. reacting, e.g. simultaneously mixing, an aqueous solution of iron(III) salt with an aqueous base at pH comprised between about 6 and 10, wherein the reaction is optionally performed in the presence of said insoluble carbohydrate (preferably starch);
(i) either adding said insoluble carbohydrate (preferably starch) if it was not present in step i)
or optionally adding more of said insoluble carbohydrate (preferably starch);
(ii) isolating the precipitate formed; and optionally washing, e.g. with water;
(iii) suspending the precipitate in an aqueous solution; and
(iv) adding a soluble carbohydrate (preferably a glucose derivative, such as sucrose or maltodextrin) to yield an iron (III)-based phosphate adsorbent.

2.
i) reacting, e.g. simultaneously mixing, an aqueous solution of iron (III) salt with an aqueous base at pH comprised between about 6 and 10, wherein the reaction is performed in the presence of said insoluble carbohydrate (preferably starch);
ii) optionally adding more of said insoluble carbohydrate (preferably starch) before the precipitation of the iron (III) is complete, e.g. has started;
   wherein steps iii) to v) are performed as defined under 1.

3.
i) reacting, e.g. simultaneously mixing, an aqueous solution of iron (III) salt with an aqueous base at pH comprised between about 6 and 10;
ii) adding said insoluble carbohydrate (preferably starch), before the precipitation of the iron (III) is complete, e.g. has started;
   wherein steps iii) to v) are performed as defined under 1.

4.
i) reacting, e.g. simultaneously mixing, an aqueous solution of iron(III) salt with an aqueous base, and
ii) either performing step i) in presence of an insoluble carbohydrate, e.g. starch, and optionally adding more insoluble carbohydrate after the complete mixing:
or adding the insoluble carbohydrate after the reaction of step i), e.g. after the complete mixing,
   wherein steps iii) to v) are performed as defined under 1.

5.
i) reacting, e.g. simultaneously mixing, an aqueous solution of iron(III) salt with an aqueous base, in presence of an insoluble carbohydrate (preferably starch), wherein the pH of the solution is maintained at a value between about 6 and 8;
   wherein steps iii) to v) are performed as defined under 1.

6. A process as defined under 1 to 5 wherein the process further comprises the step vi) of isolating the product, preferably by spray drying or fluidized spray drying to give an iron (III)-based phosphate adsorbent as a dry powder.

7. A process as defined under 1 to 6 wherein the process further comprises the step of granulating the powder, optionally in presence of at least one excipient selected from a binder and a lubricant, to yield an iron (III)-based phosphate adsorbent as a granulate.

8. A process as defined under 1 to 7 wherein the process further comprises the step viii) of tabletting either the powder obtained in step vi) or the granulate obtained in step vii), wherein the tabletting step is optionally performed in presence of an excipient selected from a filler, a binder, a disintegrant, a flow agent, a lubricant, and mixture thereof, as hereinabove described.

9. Alternatively, SBR 759 may be produced by a process which comprises the steps of
a) reacting an aqueous solution of iron (III) salt (e.g. iron (III) chloride) with a aqueous base at pH comprised between 6 and 10, wherein the reaction is optionally performed in the presence of starch;
b) adding starch if starch was not present in step a), and optionally
a) isolating the solids and washing.

SBR759 may be prepared as a powder and may be used with no additional excipients.

Alternatively, SBR759 may be formulated, for example with additional excipients, in any conventional form, preferably oral dosage form, e.g. granules, granulates, capsules, sachets, stick packs, bottles, optionally together with adequate dosing systems, e.g., calibrated spoons, tablets, dispersible tablets, chewable tablets, film coated tablets, or uniquely coated tablets.

SBR759 may also be formulated as semi-solid formulations, e.g. aqueous and non-aqueous gel, swallowable gel, chewy bar, fast-dispersing dosage, cream ball chewable dosage form, chewable dosage forms, or edible sachet.

These and further alternative forms may be obtained according to the disclosure in International patent publication WO 2008/071747 which is incorporated by reference.

In a preferred embodiment of the invention, SBR759 is prepared in the form of a powder or a granulated product, which is optionally filled into powder containers such as bottle, capsule, sachet or stick pack.

The sachet or stick packs may contain between about 0.125 to 10 g, e.g. from about 0.25 to 1, 1.5, 2 or 5 g, e.g. from about 0.25 to 1.5 g, of granulated product.

The oral iron (III)-based phosphate adsorbent, for example SBR759, for use in cats should be incorporated into cat feed each day to achieve a concentration of 5 g to 20 g per kg of complete dry feed, equivalent to a dose of 0.125 to 10 g, for example a dose of 0.125 or 0.25 g to 1, 1.5, 2, or 5 g per cat per day.

Since no change in mechanism of action (binding oral phosphate in the food and the gastrointestinal tract) is expected with time, the data justify no time limit to therapy.

Accordingly, in one aspect of the invention the oral iron (III)-based phosphate adsorbent, for example SBR759, is administered, for example with cat food, continuously throughout the cat's life.

SBR759 is well accepted by cats, due to its high palatability.

It is recommended to administer SBR759 at a starting dose of 0.125 g/day and to increase in increments of 0.125 g/day up to a maximum of 10.0 g/day, preferably to a maximum of 1, 1.5, 2 or 5 g/day. Plasma phosphate concentrations should be monitored during therapy.

The new use of the invention is described by the following embodiments of the invention which alone or in combination contribute to solving the object of the invention:
1.An oral iron (III)-based phosphate adsorbent, for example SBR759, for use in a method of treating iron-deficiency anaemia in cats with chronic kidney disease.
2.An oral iron (III)-based phosphate adsorbent, for example SBR759, for use in a method of improving of red cell variables in cats with chronic kidney disease.
3.An oral iron (III)-based phosphate adsorbent, for example SBR759, for use in a method of increasing quality of life of cats with chronic kidney disease.
4.An oral iron (III)-based phosphate adsorbent, for example SBR759, for use in a method of increasing survival in cats with chronic kidney disease.
5. A method of treating iron-deficiency anaemia in cats with chronic kidney disease which method comprises administering an oral iron (III)-based phosphate adsorbent, for example SBR759.
6.A method or an oral iron (III)-based phosphate adsorbent, for example SBR759, for use in a method according to any preceeding numbered paragraph wherein the oral iron (III)-based phosphate adsorbent, for example SBR759, is administered in a therapeutically effective amount, for example at a dose of 0.125 g to 10.0 g per cat per day, for example at a dose of 0.25 g to 1, 1.5, 2 or 5 g per cat per day.
7. A method or an oral iron (III)-based phosphate adsorbent, for example SBR759, for use in a method according to numbered paragraph 6 wherein the oral iron (III)-based phosphate adsorbent, for example SBR759, dose is mixed homogenously into the daily food ration.
8.A method or a oral iron (III)-based phosphate adsorbent, for example SBR759, for use in a method according to numbered paragraph 7 wherein the oral iron (III)-based phosphate adsorbent, for example SBR759, is administered with cat food continuously throughout the cat's life.
9. Use of an oral iron (III)-based phosphate adsorbent, for example SBR759, in the manufacture of a medicament for one or more of the following: the treatment of iron-deficiency anaemia; improving red cell variables; increasing quality of life; or increasing survival in cats with chronic kidney disease.
10. A use according to numbered paragraph 9 wherein the oral iron (III)-based phosphate adsorbent, for example SBR759, is administered in a therapeutically effective amount, for example at a dose of 0.125 g to 10.0 g per cat per day, for example at a dose of 0.25 g to 1, 1.5, 2 or 5 g per cat per day, for example wherein the oral iron (III)-based phosphate adsorbent, for example SBR759, dose is mixed homogenously into the daily food ration, for example, wherein the oral iron (III)-based phosphate adsorbent, for example SBR759, is administered with cat food continuously throughout the cat's life.
11. An oral iron (III)-based phosphate adsorbent, for example SBR759, a method or a use according to any preceding paragraph, wherein the oral iron (III)-based phosphate adsorbent, for example SBR759, is used in combination with available phosphate binders.

The following non-limiting example further illustrates the invention.

### EXAMPLE

Study: A 12-week, open label, multicenter, dose determination study to evaluate the efficacy of SBR759 as an oral phosphate binder in cats with chronic kidney disease.

Methodology: Prospective, multicenter, open label, dose determination field study in client owned cats with stable naturally occurring chronic kidney disease and hyperphosphatemia. An adaptive design was used resulting in starting doses of 0.5 and 0.125 g/day in phases I and II, with additional individual titration during 3 parts of each phase, resulting in a dose range 0.125 to 1.5 g/day.

### Investigational Veterinary Product:

SBR 759 powder packed in 0.25 and 1 g sachets. Administered orally with food at nominal doses of 0.125, 0.25, 0.5, 1 and 1.5 g/day.

### Evaluation Criteria:

Plasma concentrations of phosphate and frequency of “treatment success”. Plasma Ca x PO₄ product and PTH concentration. Subjective assessments of cats' appetite, clinical condition and quality of life. Clinical chemistry, hematology and urine analysis. Frequency of adverse events.

### Results:

Efficacy: SBR759 produced a significant reduction in plasma phosphate concentrations at doses of 0.25, 0.5, 1 and 1.5 g/day. Effects were dose-related. The benefit of SBR759 on plasma phosphate concentrations was shown in chronic kidney disease stages II, Ill and IV and in all classes of diet: renal diet, no renal diet and mixed. SBR759 was also associated with beneficial reductions in plasma Ca x PO₄ product (0.5 and 1 g/day) and plasma PTH concentrations (1 g/day), and improvement in quality of life (0.5, 1 and 1.5 g/day) and red cell variables (1 and 1.5 g/day). In most cats palatability of SBR759 was rated as good or excellent, and ease of administration was rated as easy or very easy.

Safety: There were no significant changes in plasma calcium concentrations, although there was a non-significant trend for a dose-related increase. SBR759 produced a dose-related increase in plasma iron concentrations especially at doses of 0.5 g/day and higher. This effect was apparently beneficial in this study, being associated with significant improvements in red cell variables (cell count, hemoglobin concentrations and hematocrit).

Hematology: Blood samples for hematology were taken from a vein into EDTA tubes at Visits 2, 4, 5 and 6. The samples were labelled and packaged and sent immediately in a cool box to the Central Laboratory. If the samples could not be sent on the day of collection, they were stored in a refrigerator. The following hematological parameters were measured:
Red Blood Cell count
White Blood Cell count and differential count
Platelet count
Hematocrit
Hemoglobin

Plasma iron concentrations were higher after treatment with SBR759 compared to baseline This result is attributed to absorption from the gastrointestinal tract of a small amount of iron, since SBR759 is a complex of iron and starch. Similar effects have been described in other species.

Cats with chronic kidney disease are at risk of anemia, therefore moderate intake of iron should not be harmful and may in fact be beneficial. Evidence for this benefit was observed from the fact that the mean erythrocyte count, hemoglobin concentration and hematocrit all increased with dose of SBR759.

Increases from baseline were significant (p<0.05) for red cell count and hematocrit (1 and 1.5 g/day) and hemoglobin concentration (1.5 g/day) (Table 1).

The mean hemoglobin concentration was lower (94.8 g/L) than the normal range (95-150 g/L) in the 0.5 g/day group at baseline and this had increased to within the normal range (99.2 g/L) at Visit 6 (Table 2). The mean hemoglobin concentration at baseline was also lower (88.0 g/L) than the normal range (95-150 g/L) in the 0.125 g/day group at baseline and this increased slightly to 89.60 g/L (i.e. still below the normal range) at Visit 6 (Table 2). It is concluded that no relevant increase in plasma iron concentration occurred at the 0.125 and 0.25 g/day doses of SBR759, but there were moderate increases at 0.5, 1.0 and 1.5 g/day in some cats. This moderate increase in plasma iron concentrations was associated with improvement in red cell parameters (red cell count, hematocrit and hemoglobin concentration) and therefore associated with beneficial effects.

TABLE 1
Hematology, comparison to baseline by nominal dose

| | nominal | | |
|---|---|---|---|
| Parameter | dose [g/d] | n | p-value |
| Red blood cell | 0.125 | 19 | 0.6221 |
| count [10\hat{ }12/L] | 0.25 | 16 | 0.2877 |
| | 0.5 | 36 | 0.2959 |
| | 1 | 20 | 0.0431 I |
| | 1.5 | 16 | 0.0400 I |
| Hemoglobin | 0.125 | 19 | 0.5342 |
| [g/L] | 0.25 | 16 | 0.2318 |
| | 0.5 | 36 | 0.7730 |
| | 1 | 20 | 0.1362 |
| | 1.5 | 16 | 0.0297 I |
| Hematocrit | 0.125 | 19 | 1.0000 |
| [L/L] | 0.25 | 16 | 0.8672 |
| | 0.5 | 36 | 0.6924 |
| | 1 | 20 | 0.0030 I |
| | 1.5 | 16 | 0.0034 I |

D = significant decrease,
I = significant increase

TABLE 2
Hematology, summary statistics

| | | initial dose 0.125 g | | | initial dose 0.5 g | | | all animals | | |
|---|---|---|---|---|---|---|---|---|---|---|
| parameter | visit | n | mean | s.d. | n | mean | s.d. | n | mean | s.d. |
| Red blood cell | base. | 19 | 6.85 | 1.58 | 22 | 6.57 | 1.34 | 41 | 6.70 | 1.44 |
| count | V4 | 20 | 7.02 | 1.82 | 25 | 6.62 | 1.19 | 45 | 6.80 | 1.50 |
| [10\hat{ }12/L] | V5 | 20 | 7.09 | 1.64 | 25 | 6.78 | 1.17 | 45 | 6.92 | 1.39 |
| RR: 4-9 | V6 | 20 | 7.05 | 1.70 | 25 | 6.82 | 1.28 | 45 | 6.92 | 1.47 |
| Hemoglobin | base. | 19 | 87.95 | 19.56 | 22 | 94.77 | 20.90 | 41 | 91.61 | 20.33 |
| [g/L] | V4 | 20 | 90.10 | 20.33 | 25 | 94.76 | 17.88 | 45 | 92.69 | 18.93 |
| RR: 95-150 | V5 | 20 | 91.50 | 20.07 | 25 | 96.32 | 18.71 | 45 | 94.18 | 19.25 |
| | V6 | 20 | 89.60 | 20.69 | 25 | 99.24 | 18.05 | 45 | 94.96 | 19.65 |
| Hematocrit | base. | 19 | 0.298 | 0.065 | 22 | 0.295 | 0.057 | 41 | 0.297 | 0.060 |
| [L/L] | V4 | 20 | 0.301 | 0.067 | 25 | 0.299 | 0.053 | 45 | 0.300 | 0.059 |
| RR: 0.24-0.45 | V5 | 20 | 0.303 | 0.065 | 25 | 0.307 | 0.051 | 45 | 0.305 | 0.057 |
| | V6 | 20 | 0.300 | 0.068 | 25 | 0.312 | 0.057 | 45 | 0.306 | 0.061 |

### Quality of Life

The quality of life of the cat was assessed by the owner at Visits 2, 3, 4, 5 and 6 taking into account the activity and general behavior of the cat at home.

At Visit 2 the following scheme was used:

0—very poor, 1—poor, 2—slightly impaired as compared to normal, 3—good i.e. normal.

At Visits 3, 4, 5 and 6, the change from baseline was assessed as follows:

0—worse, 1—same, 2—improved, 3—much improved.

For the secondary endpoints 4, 5 and 6, “normal” referred to “not ill”.

There was a significant increase from baseline in quality of life in the 0.5, 1 and 1.5 g/day groups (Table 3).

Mean quality of life was rated as ˜2.5 at baseline (between slightly impaired and good) and was rated between 1 and 2 (i.e. between same and improved) at visits 3 to 6 (FIG. 1).

TABLE 3
Quality of life scores, comparison to baseline, vs. nominal dose

| | nominal | | | | |
|---|---|---|---|---|---|
| | dose | all cases | | complete cases | |
| parameter | [g/d] | n | p-value | n | p-value |
| Quality of life | 0.125 | 20 | 0.0781 | 17 | 0.1563 |
| change score | 0.25 | 18 | 0.0918 | 17 | 0.1484 |
| | 0.5 | 40 | 0.0019 I | 34 | 0.0078 I |
| | 1 | 23 | 0.0137 I | 19 | 0.0039 I |
| | 1.5 | 19 | 0.0283 I | 18 | 0.0283 I |

D = significant decrease,
I = significant increase

### LITERATURE REFERENCE LIST

Block G. A., Brillhart S. L., Persky M. S., Amer A. & Slade A. J. (2010) Efficacy and safety of SBR759, a new iron-based phosphate binder. Kid Int 77: 897-903.

Chalhoub S., Langston C. E. & Farrelly J. (2012) The use of darbopoetin to stimulate erythropoiesis in anemia of chronic kidney disease in cats. J Vet Intern Med 26: 363-369.

Chakrabarti S., Syme H. M. & Elliott J. (2012) Clinicopathological variables predicting progression of azotemia in cats with chronic kidney disease. J Vet Intern Med 10.1111/j.1939-1676.2011.00874.x

Elliott J., Rawlings J. M., Markwell P. J. & Barber P. J. (2000) Survival of cats with naturally occurring chronic renal failure: effect of dietary management. J Small Anim Pract 41: 235-242.

Geisser P. & Philipp E. (2010) PA21: a novel phosphate binder for the treatment of hyperphosphatemia in chronic kidney disease. Clinical Nephrology, Vol. 74—No. 1 (4-11) Hergesell O. & Ritz E. (1999a) Phosphate binders on an iron basis: a new perspective? Kid Int Suppl 73: S42-S45.

Isakova T., Gutiérrez M., Chang Y., Shah A., Tamez H., Smith K., Thadhani R. & Wolf M. (2009) Phosphorus binders and survival on hemodialysis. J Am Soc Nephrol 20: 388-396.

King J. N., Tasker S., Gunn-Moore D., Strehlau G., and the BENRIC (Benazepril in Renal Insufficiency in Cats) Study Group (2007) Prognostic factors in cats with chronic kidney disease. J Vet Intern Med 21: 906-916.

Kuntz E., Hall, C., Dip, R. & King, J. N. (2012) Three-month tolerance of the novel oral phosphate binder SBR759 in cats. J Vet Pharmacol Therap 35 (Supplement 3): 171.

McIntyre C. W., Pearl P., Warwick G., Wilkie M., Toft A. J. & Hutchison A. J. (2009) Iron-magnesium hydroxycarboante (Fermagate): A novel non-calcium-containing phosphate binder for the treatment of hyperphosphatemia in chronic hemodialysis patients. Clin J Am Soc Nephrol 4: 401-409.

Nunamaker E. A. & Sherman J. G. (2011) Oral administration of lanthanum dioxycarbonate does not alter bone morphology of normal cats. J Vet Pharmacol Therap 35: 193-197.

Sinsakul M., Sika M., Koury M., Shapiro W., Greene T., Dwyer J., Smith M., Korbet S. & Lewis J., (2012) The safety and tolerability of ferric citrate as a phosphate binder in dialysis patients. Nephron Clin Pract 121:c25-c29.

Speranza C., Seewald W., Dip, R., Schmid, V. B. & King J. N. (2012) Field efficacy and tolerability of the novel oral phosphate binder SBR759 in cats. J Vet Pharmacol Therap 35 (Supplement 3): 171.

Tonelli M., Pannu N. & Manns B. (2010) Oral phosphate binders in patients with kidney failure. New Eng J Med 362: 1312-1324.

Wüthrich R. P., Chonchol M., Covic A., Gaillard S., Chong E. & Tumlin J. A. (2012) Randomized clinical trial of the iron-based phosphate binder PA21 in hemodialysis patients. CJASN ePress, Clin J Am Nephrol 8: xxx-xxx, 2013

Yokoyama K., Hirakata H., Akiba T., Sawada K., & Kumagai Y. (2012) Effect of oral JTT-751 (ferric citrate) on hyperphosphatemia in hemodialysis patients: results of a randomized, double-blind, placebo-controlled trial. Am J Nephrol 36:478-487.

1-3. (canceled)

4. A method of treating iron-deficiency anaemia in cats with chronic kidney disease which method comprises administering SBR759.

5. (canceled)

6. The method according to claim 4 wherein SBR759 is administered at a dose of 0.125 g to 1.5 g per cat per day.

7. The method according to claim 6 wherein the SBR759 dose is mixed homogenously into the daily food ration.

8. The method according to claim 7 wherein SBR759 is administered with cat food continuously throughout the cat's life.

9-11. (canceled)
